(19) United States
(12) Patent Application Publication
(10) Pub. No.: US 2025/0183716 A1
(43) Pub. Date: Jun. 5, 2025

(54) HIGH EFFICIENCY WIRELESS POWER TRANSFER TO IMPLANTABLE DEVICES

(71) Applicant: Resonant Link, Inc., South Burlington, VT (US)

(72) Inventors: Aaron Stein, Essex, VT (US); Yi Dou, Gentofte (DK); Panteleimon Papamanolis, Zurich (CH); Charles R. Sullivan, West Lebanon, NH (US); Phyo Aung Kyaw, Winooski, VT (US)

(73) Assignee: Resonant Link, Inc., South Burlington, VT (US)

(21) Appl. No.: 19/044,197

(22) Filed: Feb. 3, 2025

Related U.S. Application Data
(63) Continuation of application No. PCT/US2023/029492, filed on Aug. 4, 2023.
(60) Provisional application No. 63/397,439, filed on Aug. 12, 2022.

(30) Foreign Application Priority Data
Aug. 5, 2022 (GR) 20220100654

(51) Int. Cl. H02J 50/10; A61B 5/00

(52) U.S. Cl. CPC H02J 50/10; A61B 5/686; A61B 2560/0219; A61B 2560/0443

(57) ABSTRACT

An implantable device includes a sealed case forming an enclosure, a wireless power transfer coil within the enclosure, a battery within the enclosure, and electronics within the enclosure. The electronics may be configured to process power received through the wireless power transfer coil to charge the battery. At least one region of the sealed case may be configured to impede eddy currents.

### CROSS-REFERENCE TO RELATED APPLICATIONS

This application claims priority to Greek patent application 20220100654, filed Aug. 5, 2022, titled “HIGH EFFICIENCY WIRELESS POWER TRANSFER TO IMPLANTABLE DEVICES,” and U.S. provisional application 63/397,439, filed Aug. 12, 2022, titled “HIGH EFFICIENCY WIRELESS POWER TRANSFER TO IMPLANTABLE DEVICES,” each of which is hereby incorporated by reference in its entirety.

### BACKGROUND

### 1. Technical Field

The apparatus and techniques described herein relate to wireless power transfer.

### 2. Discussion of the Related Art

A wireless power transfer system can transfer energy wirelessly through magnetic coupling. Wireless power transfer systems may be used to power and/or charge medical devices such as implants (also termed herein “implantable devices”).

A wireless power transfer system includes a wireless power transmitter and a wireless power receiver, each of which includes a power transfer coil (also herein termed “coil” or “winding”), which may be a transmit coil or a receive coil, respectively. A wireless power transmitter may include a transmit coil that may be coupled to a power source via power electronics. The power electronics may invert a DC (direct current) signal into an AC (alternating current) signal that can be transmitted wirelessly to a wireless power receiver through electromagnetic induction. A wireless power receiver may include a receive coil and power electronics (e.g., a rectifier) that couples the receive coil to a load, such as circuitry for charging a battery. In operation, a wireless power transmitter and receiver are physically separated from one another by some distance, and the wireless power transmitter inductively transfers power to the wireless power receiver.

### SUMMARY

Some aspects relate to an implantable device, comprising: a sealed case forming an enclosure; a wireless power transfer coil within the enclosure; a battery within the enclosure; and electronics within the enclosure. The electronics may be configured to process power received through the wireless power transfer coil to charge the battery. At least one region of the sealed case may be configured to impede eddy currents.

The at least one region may be above and/or below the wireless power transfer coil.

The at least one region may have an electrical resistivity of 1 micro-ohm meter or greater.

The at least one region may include a titanium material.

The titanium material may comprise grade 5 titanium.

The at least one region may be a first at least one region that comprises at least one slit or window of higher electrical resistance than a second at least one region of the sealed case.

The first at least one region may be thinner than the second at least one region.

The first at least one region may have a higher electrical resistivity than that of the second at least one region.

The implantable device may be configured to power a second device outside of the sealed case through a wired or wireless connection.

The electronics may further comprise bioelectronics circuitry.

Some aspects relate to an implantable device, comprising: a sealed titanium case forming an enclosure; a wireless power transfer coil within the enclosure; a battery within the enclosure; and electronics within the enclosure. The electronics may be configured to process power received through the wireless power transfer coil to charge the battery.

At least one region of the sealed case may be configured to impede eddy currents.

The at least one region may be above and/or below the wireless power transfer coil.

The at least one region may have an electrical resistivity of 1 micro-ohm meter or greater.

The at least one region may comprise a titanium material.

The titanium material may comprise grade 5 titanium.

The at least one region may be a first at least one region that comprises at least one slit or window of higher electrical resistance than a second at least one region of the sealed titanium case.

The first at least one region may be thinner than the second at least one region.

The first at least one region may have a higher electrical resistivity than that of the second at least one region.

The implantable device may be configured to power a second device outside of the sealed case through a wired or wireless connection.

The electronics may further comprise bioelectronics circuitry.

Some aspects relate to a power module for an implantable device, comprising: a battery lacking a dedicated metal case; a wireless power transfer coil positioned over the battery; and electronics configured to process power received through the wireless power transfer coil to charge the battery.

The power module may be configured to power a second device outside of the implantable device through a wired or wireless connection.

Some aspects relate to a method of operating an implantable device, the implantable device having a sealed case forming an enclosure. a wireless power transfer coil within the enclosure, a battery within the enclosure, and electronics within the enclosure, wherein at least one region of the sealed case is configured to impede eddy currents, the method comprising: processing, by the electronics, power received through the wireless power transfer coil to charge the battery.

The foregoing summary is provided by way of illustration and is not intended to be limiting.

### BRIEF DESCRIPTION OF DRAWINGS

In the drawings, each identical or nearly identical component that is illustrated in various figures is represented by a like reference character. For purposes of clarity, not every component may be labeled in every drawing. The drawings are not necessarily drawn to scale, with emphasis instead being placed on illustrating various aspects of the techniques and devices described herein.

FIG. 1A shows a side view and FIG. 1B shows a top view of an implant, according to some embodiments.

FIG. 2 shows an example in which the case of an implant has thinned regions R1 and R2 which have higher electrical resistance due to their decreased thickness, thus impeding the flow of eddy currents in regions R1 and R2.

FIG. 3A shows an example of a top view of an implant case having a strip of relatively high electrical resistance extending along a radial direction. FIG. 3B shows an example of a top view of an implant case having a plurality of strips of increased electrical resistance. FIG. 3C shows an example of a top view of an implant case having windows of increased resistance.

FIG. 4 shows cross-sectional view of an example of an implant in which a wireless power transfer coil is positioned above a battery.

FIG. 5 shows an example of a wireless power implant that includes an implant electronics module including a battery, electronics and a wireless power transfer coil within a case.

### DETAILED DESCRIPTION

With an implant, high power losses are unacceptable as they are transformed into heat, which can cause an undesirable temperature rise in surrounding tissue. It would be desirable to improve or maximize the efficiency of wireless power transfer to such devices. The performance of a wireless power transfer system may be constrained by the magnetic coupling factor (k) between the coils and the quality factor (Q) of the coils. The magnetic coupling factor (k) may be limited by the wireless gap, so achieving high Q can often help achieve high-performance wireless power transfer.

The inventors have recognized and appreciated that conventional medical implants may be encased in a material that is not suitable for wireless power transfer. For example, many medical implants may have a sealed (e.g., hermetically sealed) metal (e.g., titanium) case. Transferring power wirelessly through the case to a wireless power receive coil within the case can result in eddy currents being generated in the case, which increases power losses in the case and reduces the Q, causing heating of surrounding tissue.

To reduce losses and increase Q, the case may be configured to impede eddy currents. For example, the case may be formed, in whole or in part, of regions of increased electrical resistance (resistance) to impede eddy currents in one or more locations. Such regions of increased electrical resistance may be thinner regions of the case or regions of material with higher resistivity, for example.

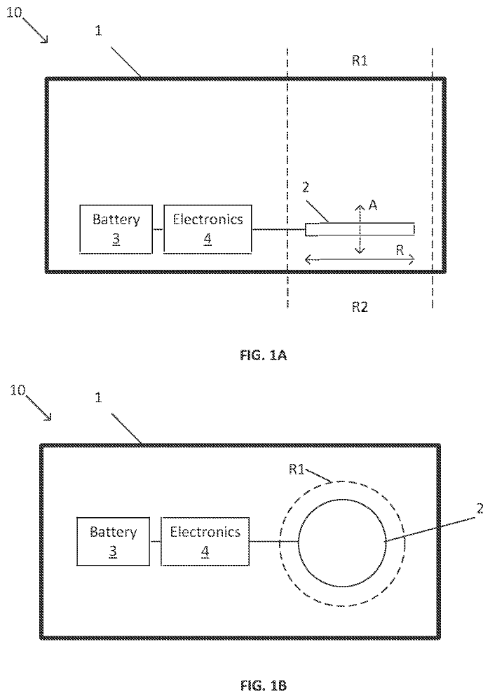

FIG. 1A shows a side view and FIG. 1B shows a top view of an implant 10, according to some embodiments. The implant may have a sealed case 1 housing a wireless power transfer coil 2, a battery 3, and electronics 4 configured to charge the battery 3 from the power received via the wireless power transfer coil 2. The electronics 4 may include a rectifier for rectifying the alternating current induced in wireless power transfer coil 2 into direct current, and/or the electronics 4 may include charging circuitry for charging the battery 3 using the rectified current. Optionally, the electronics 4 may additionally include bioelectronics circuitry configured to interface with an organism to perform any suitable bioelectronics functions (e.g., sensing and/or stimulation). The electronics 4 may be completely or partially powered by the battery 3, or not powered by battery 3. The battery 3 may be positioned to the side of the wireless power transfer coil 2, as illustrated in FIG. 1A. In some embodiments, the region R1 above the wireless power transfer coil 2, the region R2 below the wireless power transfer coil 2, or both, may be particularly prone to the inducement of eddy currents due to the magnetic flux passing (in approximately a vertical direction of FIG. 1A) through the wireless power transfer coil 2. The regions R1 and R2 are above and below the wireless power transfer coil 2, 10 respectively, in the sense that the wireless power transfer coil 2 has an axis A parallel (or antiparallel) to the direction in which magnetic flux passes through the center of the wireless power transfer coil, and the regions R1 and R2 are displaced from the wireless power transfer coil along the direction of the axis A and at least partially overlap with the wireless power transfer coil along the horizontal dimension of FIG. 1A. The horizontal dimension of FIG. 1A 15 corresponds to a radial dimension R of the wireless power transfer coil. In some embodiments, measures may be taken to reduce the eddy currents in regions R1 and/or R2.

In some embodiments, a case for an implant (e.g., case 1) may be formed, in whole or in part, of a material having relatively high electrical resistivity to reduce eddy currents. For example, such a material may have an electrical resistivity of greater than or equal to 1 micro-ohm meter. In some embodiments, the case 1 may be formed of a metal material, such as a titanium alloy. For example, if the case 1 is formed of a titanium alloy, the titanium alloy may be grade 5 titanium or an alloy with a higher resistivity. Grade 5 titanium corresponds to an alloy of titanium in which titanium is alloyed with 6% aluminum and 4% vanadium (also known as TI 6Al-4V), approximately.

In some implants the case 1 may be formed of more than one material. The regions of the case 1 that have the most effect on wireless power transfer are the regions above and/or below the wireless power transfer coil. Accordingly, in some embodiments, the portions of the case 1 in regions R1 and/or R2 (FIGS. 1A and 1B) may include a material with a relatively high electrical resistivity, such as greater than 0.01, greater than 0.1 or greater than 1 micro-ohm meter, such as greater than 10 or 100 micro-ohm meter. The electrical resistivity may be the same as or less than that of an electrical insulator (e.g., glass, polymer, etc.). The remaining portions of the case 1 may be formed of the same material or a different material. The regions R1 and/or R2 may be formed of a metal material or another material, such as ceramic, polymer, etc. In some cases, a case 1 may have a “window” of a material in the regions R1 and/or R2 that is different from other portions of the case. For example, a piece of high resistivity material (e.g., ceramic or polymer) may be attached in regions R1 and/or R2, covering and sealing an opening in a metal case in such regions.

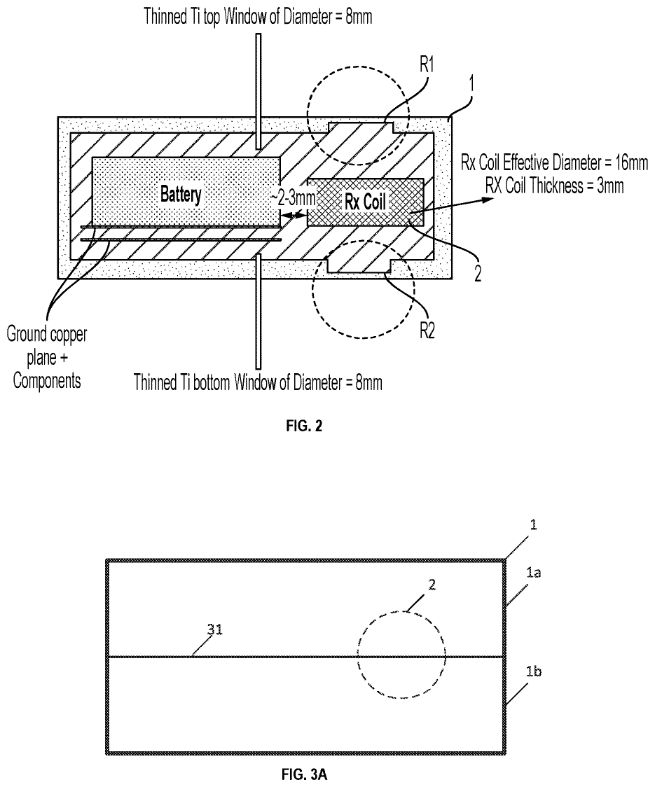

Alternatively or additionally, in some embodiments a case 1 (e.g., a metal case) may be made thinner at regions R1 and/or R2. FIG. 2 shows an example in which the case 1 has thinned regions R1 and R2 which have higher electrical resistance due to their decreased thickness, thus impeding the flow of eddy currents in regions R1 and R2. In other examples, such a technique may be applied to only region R1 or region R2. FIG. 2 also shows examples of particular thicknesses and dimensions, as well as components within the case. However, these are examples, and the devices described herein are not limited to particular thickness, dimensions or components.

The wireless power transfer coil 2 may have a greater, equal or lesser horizontal extent (in the sense of FIG. 1A) than the regions R1 and/or R2. FIGS. 1A and 1B show regions R1 and R2 having a greater horizontal extent than the wireless power transfer coil 2. FIG. 2 shows regions R1 and R2 having a lesser horizontal extent than the wireless power transfer coil 2. In other embodiments, regions R1 and R2 may have an equal horizontal extent in comparison to the wireless power transfer coil 2. The regions R1 and R2 may have any shape, and may be centered or not centered with respect to the wireless power transfer coil 2. In some embodiments where the wireless power transfer coil 2 has a magnetic core, it may be sufficient to reduce the eddy currents only on one side: the same side of the case as the wireless power transmit coil that transmits power to the implant. In embodiments without a magnetic core for the wireless power transfer coil 2, magnetic flux may pass through both regions R1 and R2, so it may be more effective to reduce eddy currents in both regions R1 and R2.

The inventors have recognized and appreciated that eddy currents may be induced to flow in a circular direction on the top surface and/or the bottom surface of the case. In some embodiments, the case may include one or more “strips” of relatively high electrical resistance between respective conductive portions of the case. The high electrical resistance may be provided by a high resistivity material and/or thinner regions of the case, for example. In some embodiments, the one or more strips may extend in a radial direction (with respect to the wireless power transfer coil), or another direction, to impede the flow of current in a circumferential direction (with respect to the wireless power transfer coil).

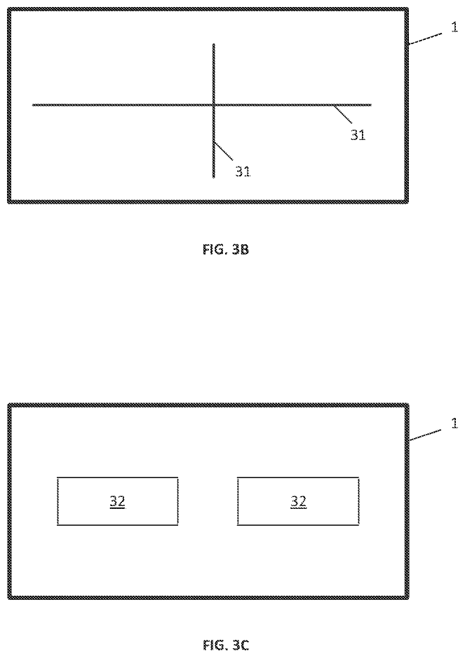

FIG. 3A shows an example of a top view of a case 1 having a strip 31 of relatively high electrical resistance extending along a radial direction. In this example, two “halves” 1a, 1b of a high conductivity (e.g., metal) case may be joined together to form the sealed enclosure, and a region of relatively low electrical conductivity material (strip 31) may be located between the two halves 1a, 1b to inhibit the flow of electrical current between the two halves 1a, 1b. In some embodiments, the strip 31 may extend across a top and/or bottom of the case, as shown in FIG. 3A, to reduce the flow of eddy currents in the top and/or bottom of the case. Alternatively or additionally, the “strip” may be a region of the case having a reduced thickness, similar to what is shown in FIG. 2. In some embodiments, a plurality of strips 31 of increased electrical resistance may be formed on a top and/or a bottom surface of the case 1, as illustrated in FIG. 3B. The region of increased resistance need not be in the shape of a narrow slit, and may any shape, such as a more two-dimensional shape, termed a “window.” An example of “windows” 32 of increased resistance (e.g., due to reduced thickness or a relatively high resistivity material) that may prevent eddy currents in a circumferential direction is shown in FIG. 3C. Such windows 32 may be formed on the top or bottom of the case or at any other locations on the case. However, these are examples, and there may be any number of slits or windows having any of a variety of positions, shapes, orientations, etc.

In some implementations it may be desirable to position the wireless transfer coil above or below a battery of an implant. FIG. 4 shows a cross-sectional view of an example of an implant 40 in which a wireless power transfer coil 2 is positioned above the battery 3. The inventors have recognized and appreciated that conventional batteries for implants include a battery case that is formed of a high conductivity material. When a wireless power transfer coil 2 is positioned above or below such a battery, eddy currents may be induced in the battery's case. In some embodiments, using an unpackaged battery can avoid the formation of eddy currents in the such a case. Accordingly, in the embodiment of FIG. 4 the battery 3 may be an unpackaged battery (e.g., without a dedicated sealed metal case). The unpackaged battery ultimately may be packaged in a sealed case 1 with other components of an implant 40.

As mentioned above, an implantable device as described herein may include a wireless power transfer coil 2 for receiving power wirelessly through magnetic induction, as well as a battery 3 and suitable electronics 4 for charging the battery 3 from the power received through the wireless power transfer coil 2. In some circumstances these components may be provided as an implant electronics module without a case, and may be provided in any configuration such as with the wireless power transfer coil 2 to the side of the battery 3 (as shown in FIG. 1A) or above or below the battery 3, as shown in FIG. 4. Such a module may then be integrated or packaged in a case along with any other desired components by a medical device provider, manufacturer, or other entity. Accordingly, some embodiments are directed to the electronics module itself, and a case is not required.

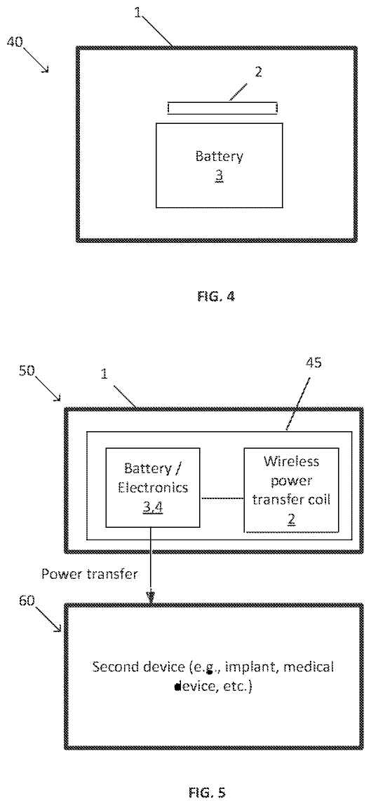

An implantable device having the electronics module described herein may or may not include additional electronics or components. In some embodiments, bioelectronics circuitry and/or components may be provided within the same case, and may be powered by the battery. In other embodiments, bioelectronics circuity may be omitted. For example, an implantable device may be provided for the purpose of receiving power wirelessly from outside the body, and for providing power to another implant or other device through a wired connection. FIG. 5 shows a block diagram of an example of such an implementation, where the electronics of a wireless power implant 50 provides power via a wired (or otherwise electrically conductive) or wireless power connection to a second device 60, which may be an implant, medical device, or another device. FIG. 5 shows wireless power implant 50 includes an implant electronics module 45 including battery 3, electronics 4 and wireless power transfer coil 2 within a case 1. Second device 60 may be a separate device from wireless power implant 1 (outside the case 1 of wireless power implant 1) having its own case.

Having described several examples of implants, examples of wireless power transfer coils 2 will be described.

A wireless power transfer coil 2 may be formed of any type of conductors, including, but not limited to: litz wire, PCB traces, foil, magnet wire, conductors laminated on substrate layers, inductively coupled current loops, multilayer self-resonant structures, electrode layers in multilayer ceramic capacitor (MLCC) processes, electrode layers in low-temperature co-fired ceramic (LTCC) processes, integrated circuit traces and others. The conductors may be planar or non-planar. Examples of non-planar coils include solenoids and barrel-wound coils. The coil or winding may be placed in a magnetic core, but placing them in a magnetic core is optional.

If the wireless power transfer coil 2 has a magnetic core, the magnetic core may be, wholly or partially, made of one or more ferromagnetic materials, which have a relative permeability of greater than 1, optionally greater than 10. The magnetic core materials may include, but are not limited to, one or more of iron, various steel alloys, cobalt, ferrites including manganese-zinc (MnZn) and/or nickel-zinc (NiZn) ferrites, nanogranular materials such as Co—Zr—O, and powdered core materials made of powders of ferromagnetic materials mixed with organic or inorganic binders. However, the techniques and devices described herein are not limited as to the particular material of the magnetic core. The shape of the magnetic core may be: a pot core, a sheet (I core), a sheet with a center post, a sheet with an outer rim, RM core, P core, PH core, PM core, PQ core, E core, EP core, or EQ core, by way of example. However, the techniques and devices described herein are not limited to the particular magnetic core shape.

The battery 3 may use any suitable battery chemistry. In some embodiments, the battery may be a metal ion battery, such as a lithium ion battery, for example.

In some embodiments, wireless power transfer to implants may be performed at a frequency below 1 MHz, but is not limited to such frequencies, as in other embodiments wireless power transfer may be at a frequency above 1 MHz (e.g., in the 6.78 MHz band).

The term “titanium case” refers to a case having at least one region that consists essentially of titanium or a titanium alloy. Such a case may have one or more windows of a different material, such as a non-conductive material (e.g., ceramic material). Such window(s) may be located above and/or below the wireless power transfer coil, as mentioned above.

Any of the techniques described herein may be used alone or in any suitable combination.

Various aspects of the apparatus and techniques described herein may be used alone, in combination, or in a variety of arrangements not specifically discussed in the embodiments described in the foregoing description and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “substantially,” “approximately,” “about” and the like refer to a parameter being within 25%, optionally within 10%, optionally less than 5% of its stated value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

1. An implantable device, comprising:
a sealed case forming an enclosure;
a wireless power transfer coil within the enclosure;
a battery within the enclosure; and
electronics within the enclosure, the electronics being configured to process power received through the wireless power transfer coil to charge the battery,
wherein at least one region of the sealed case is configured to impede eddy currents.

2. The implantable device of claim 1, wherein the at least one region is above and/or below the wireless power transfer coil.

3. The implantable device of claim 1, wherein the at least one region has an electrical resistivity of 1 micro-ohm meter or greater.

4. The implantable device of claim 1, wherein the at least one region comprises a titanium material.

5. The implantable device of claim 4, wherein the titanium material comprises grade 5 titanium.

6. The implantable device of claim 1, wherein the at least one region is a first at least one region that comprises at least one slit or window of higher electrical resistance than a second at least one region of the sealed case.

7. The implantable device of claim 6, wherein the first at least one region is thinner than the second at least one region.

8. The implantable device of claim 6, wherein the first at least one region has a higher electrical resistivity than that of the second at least one region.

9. The implantable device of claim 1, configured to power a second device outside of the sealed case through a wired or wireless connection.

10. (canceled)

11. An implantable device, comprising:
a sealed titanium case forming an enclosure;
a wireless power transfer coil within the enclosure;
a battery within the enclosure; and
electronics within the enclosure, the electronics being configured to process power received through the wireless power transfer coil to charge the battery.

12. The implantable device of claim 11, wherein at least one region of the sealed titanium case is configured to impede eddy currents.

13. The implantable device of claim 12, wherein the at least one region is above and/or below the wireless power transfer coil.

14. The implantable device of claim 12, wherein the at least one region has an electrical resistivity of 1 micro-ohm meter or greater.

15. The implantable device of claim 12, wherein the at least one region comprises a titanium material.

16. (canceled)

17. The implantable device of claim 12, wherein the at least one region is a first at least one region that comprises at least one slit or window of higher electrical resistance than a second at least one region of the sealed titanium case.

18. The implantable device of claim 17, wherein the first at least one region is thinner than the second at least one region.

19. The implantable device of claim 17, wherein the first at least one region has a higher electrical resistivity than that of the second at least one region.

20. The implantable device of claim 11, configured to power a second device outside of the sealed titanium case through a wired or wireless connection.

21. (canceled)

22. A power module for an implantable device, comprising:
a battery lacking a dedicated metal case;
a wireless power transfer coil positioned over the battery; and
electronics configured to process power received through the wireless power transfer coil to charge the battery.

23. The power module of claim 22, configured to power a second device outside of the implantable device through a wired or wireless connection.

24. (canceled)
